(19) United States
(12) Patent Application Publication
(10) Pub. No.: US 2008/0102035 A1
(43) Pub. Date: May 1, 2008

(54) Medical device having discrete regions

(76) Inventors: Vipul Bhupendra Dave, Hillsborough, NJ (US); Chao Chen, Edison, NJ (US); Jason Allan Massey, Oakville (CA); David G. Cook, Erin (CA); Jacob Leidner, Toronto (CA)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/590,405

(22) Filed: Oct. 31, 2006

(51) Int. Cl. A61K 49/04; A61F 2/06

(52) U.S. Cl. 424/9.4; 424/423; 604/890.1; 623/1.11

(57) ABSTRACT

An apparatus prepares a medical device from a polymeric composition having properties such as increased visibility of the device and the ability to deliver therapeutic and other agents. The device is constructed with additives incorporated into the polymeric structure, such as a material that increases visibility of the device, while still maintaining desired mechanical characteristics such as high radial stiffness, minimized recoil values, and improved flexibility. The device can assume a wide range of geometries that are adaptable to various loading conditions In order to include performance-enhancing additives to the medical device without affecting mechanical performance, the additives are localized in discrete regions of a polymer structure from which a medical device will be formed. For example, a medical device can be prepared from a polymer form such as a tube containing radiopaque agent localized at its ends or in a desired pattern along the device.

### FIELD OF THE INVENTION

The present invention relates to methods for making polymeric medical devices, such as intraluminal polymeric drug-eluting stents. In particular, devices are formed from polymers blended with materials that are localized within regions of the polymeric device minimizing impact on the mechanical performance of the device.

### BACKGROUND OF THE INVENTION

Medical devices can be constructed from biodegradable materials such as polymers. For example, a stent constructed from a biodegradable polymer is a medical device that is implanted into a vessel. The stent exerts an acute and/or chronic outward force that will help, to remodel a vessel to its intended luminal diameter. The stent may also contain a therapeutic agent that is delivered to the vessel at a desired location.

Forming a medical device from bioabsorbable polymers must be accomplished in such a manner as to insure that the device maintains patency when implanted into a vessel or other conduit within a body. For example, a polymeric stent is typically implanted into a vessel by expansion with a balloon or some other expandable means. It is crucial to ensure that the stent impinges upon the inner wall of the vessel. After expansion, however, the polymer stent will experience shrinkage or recoil that causes it to lose apposition. The performance of the polymeric device may be enhanced utilizing certain polymer blends and additives to achieve desired mechanical properties. The blends and additives help to prevent excessive radial recoil upon deployment, exhibit sufficient fatigue resistance and exhibit sufficient ductility so as to provide adequate coverage over the full range of intended expansion diameters.

Maximizing the performance of the polymeric medical device also requires that it be accurately placed. For example, a drug-eluting polymeric stent must be placed within a vessel at the diseased site. If the stent is mis-placed, the diseased site will not be properly treated and will require the implantation of a new stent. Placement of a new stent, however, could be complicated by the presence of the previously misplaced stent. Thus, it is desired for at least part of the device to be visible from outside the patient to ensure proper placement. X-rays are one monitoring means employed to determine the position of a medical device as it is being implanted within a patient. Radiopaque additives, which are visible by X-Ray, can be dispersed throughout a polymeric device.

In order to achieve adequate radiopacity as much as 20%-30% by weight of radiopaque agent may be required. The presence of the radiopaque agent dispersed throughout the tube can affect the mechanical properties of the polymeric device such as making the device too brittle. Moreover, incorporating radiopaque agent directly within the polymeric device complicates the manufacturing process. One alternative to incorporating the radiopaque agent directly into the polymer(s) used to construct the device is to place maker bands directly on the device at a desired location. The drawback to this approach, however, is that radiopaque agents tend to be metallic and do not easily bond onto polymeric structures. Thus, the link between the radiopaque agent and device will be purely mechanical and may not exhibit sufficient strength to withstand implantation.

Medical devices may contain a therapeutic agent that further ensures proper modeling of a conduit, such as a vessel, by preventing restenosis or neointimal hyperplasia. Polymeric devices improve the delivery of the therapeutic drug and are formed such that the drug is dispersed within the polymer matrix. In order to place the drug within the polymer matrix a solvent may be employed. The removal of the solvent causes the polymer to assume a structure that can adversely affect the mechanical performance of the device. One solution is to coat the device with the drug such that the polymer is not affected. A coated device, however, does not provide the same level of drug elution as a device having drug dispersed within the polymer matrix.

Currently, there is no polymeric medical device or method for making a polymeric medical device wherein the mechanical performance is not adversely affected by the addition to the medical device of materials that enhance visibility and therapeutic agents.

### SUMMARY OF THE INVENTION

A medical device is constructed from a unique polymeric composition having properties such as increased visibility of the device and the ability to deliver therapeutic and other agents. The device is constructed with additives incorporated into the polymeric structure, such as a material that increases visibility of the device, while still maintaining desired mechanical characteristics such as high radial stiffness, minimized recoil values, and improved flexibility. The device can assume a wide range of geometries that are adaptable to various loading conditions and may be utilized for any number of medical applications including vessel patency devices, such as vascular stents, biliary stents, ureter stents, vessel occlusion devices such as atrial septal and ventricular septal occluders, patent foramen ovale occluders and orthopedic devices such as fixation devices.

In order to include performance-enhancing additives to the medical device without affecting mechanical performance, the additives are localized in discrete regions of a polymer structure from which a medical device will be formed. For example, a medical device can be prepared from a polymer form such as a tube containing radiopaque agent localized at its ends. The polymer tubes have discrete transition regions demarcating the radiopaque agent from the regions not containing the agent but do not exhibit dimensional discontinuity. The method for forming the polymer tubes produces different compositions in different segments of the tube including varying the loading of a therapeutic agent along the length of the device formed from the tube.

More particularly, polymer structures with and without an agent are formed, sized, and then assembled by bonding appropriate segments. The bonding process employs a bonding or gluing agent. Other bonding or sealing processes may be employed such as localized heating and solvent vapor bonding. The localized heating process is performed within a short time period and in a small local area to minimize detrimental effects on therapeutic agents if present. Alternatively, an agent can be placed on a mandrel or other substrate. A polymer form advanced onto the substrate in such a way as to bring a discrete region of the form into contact with the agent. The agent is compressed between a stop and the form. After a length of time, the agent bonds with the polymer. Other approaches include laying down a coating on a mandrel or substrate using a nozzle, advancing a polymer form along the longitudinal length of the mandrel, toward the nozzle, and removing the nozzle when it comes into contact with the polymer form.

Tubes with and without an agent are prepared using either melt extrusion or solvent casting. The tubes are cut into desired dimensions depending upon the device to be formed there from. The tubes are then placed on a mandrel to form the localized regions. The order of placing the tube onto the mandrel or the manner in which it is cut is determined by the desired region in which the agent will be localized. For example, if it is desired to have a radiopaque agent at an end of a device, the tube containing the agent is placed on the mandrel first followed by the tube without the agent. Alternatively, if it is desired to have the radiopaque marker confined to a region along the longitudinal length of the device, then a portion of the tube without the agent can removed and replaced with a section of the tube containing the agent. Other patterns and designs for the localized regions can be formed in a similar manner as will be appreciated by one of skill in the art.

Once the tubes or sections of the tubes are aligned on the mandrel, they are bonded together. The tube is then formed into a medical device by a process such as laser cutting. Alternatively, the tubes with and without agent can be formed into medical devices. The completed devices can then be cut or sized so that discrete sections of the device with and without the agent can be bonded together.

An apparatus for carrying out the steps of making the medical device includes a mandrel for mounting the tubes thereon, a dispensing device for providing bonding agent, a gripping device for holding polymer forms onto the mandrel and pressing them together, and a controller which allows for manual and automatic actuation of the process for joining the polymer forms together. As described above, at least two polymer forms are mounted onto the mandrel. The mandrel is mounted onto a block such that it can be rotated or moved longitudinally. This allows for bonding agents to be applied to a location on the mandrel where the at least two tubes will be joined. After the bonding agent is applied the tubes are brought into tight contact.

The bonding solution is deposited onto the mandrel using the dispensing device. The device is extended into position using a guide such as a pneumatic cylinder. The dispensing device can be adjusted with varying degrees of freedom to achieve proper location over the mandrel where solution will be deposited. The amount of bonding solution dispensed depends on a dispense controller which governs the amount of bonding agent dispensed. After bonding agent is placed on the mandrel, the gripping device is actuated to pull together the tube sections on the mandrel. In one embodiment, the gripping device comprises fingers connected to weights through a pulley system. The load is applied to the polymer forms when a pneumatic cylinder is retracted allowing the fingers to be pulled together on a linear guide. Fittings located on the mandrel engage the fingers to clamp the tubes sections together.

A wiping assembly removes excess bonding agent from the polymer forms. The wiping assembly comprises several rollers through which a wiping surface such as a film is pulled. The film is dispensed from one roller through an idle roller and wound onto another driven roller. As the film is pulled over the rollers, a pneumatic cylinder lifts the front roller so that the film is pushed into contact with the mandrel and absorbs any excess bonding solution. The film contacts as it is pulled through the rollers wiping off the excess bonding solution.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1A is a side view of tube constructed from at least on polymeric material in accordance with the present invention.

FIG. 1B is side view of a tube constructed from at least one polymeric material having at least one additive in accordance with the present invention.

FIG. 2 is a side view of a tube constructed from the tubes having the materials of FIGS. 1A and 1B.

FIG. 3 is a side view of a tube constructed from the tubes having the materials of FIGS. 1A and 1B.

FIG. 4 is a planar view of a medical device constructed from the tube of FIG. 2.

FIG. 4A is a planar view showing the medical device of FIG. 4 in greater detail.

FIG. 5 is a planar view of a section of a medical device constructed from the tube of FIG. 3.

FIG. 6 is a front view of an apparatus for constructing a tube in accordance with the present invention.

FIG. 7 is a planar view of the apparatus of FIG. 6.

### DETAILED DESCRIPTION OF THE INVENTION

Implantable medical devices may be fabricated from any number of suitable biocompatible materials, including materials such as polymeric materials. The internal structure of these polymeric materials may be altered utilizing mechanical and/or chemical manipulation. These modifications may be utilized to create devices having specific characteristics such as crystalline and amorphous morphology and orientation.

In accordance with the present invention, implantable medical devices may be fabricated from any number of biocompatible polymeric materials. These polymeric materials may be non-degradable, biodegradable and/or bioabsorbable. These polymeric materials may be formed from single polymers, blends of polymers and blends of polymers and plasticizers. In addition, other agents such as drugs and/or radiopaque agents may be blended with the polymeric materials or affixed or otherwise added thereto. A number of chemical and/or physical processes may be utilized to alter the chemical and physical properties of the materials and ultimately the final devices.

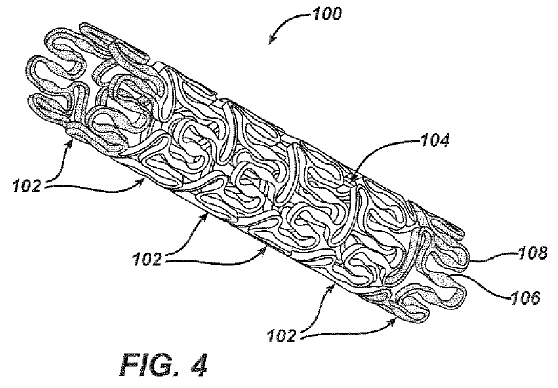

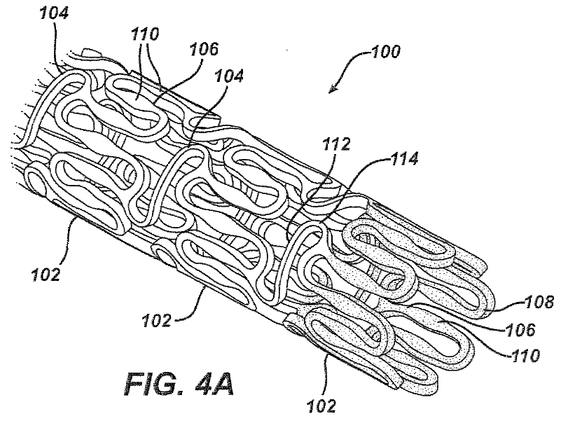

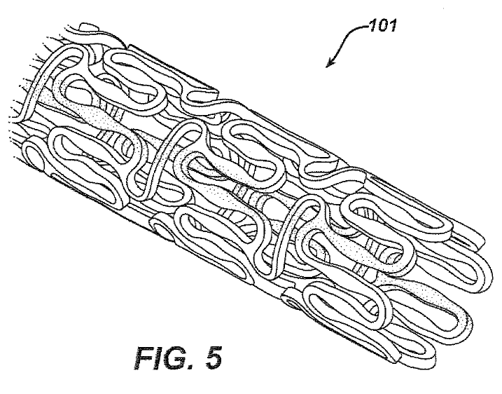

One example of a medical device that can be manufactured from the materials described above is a stent. A stent is commonly used as a tubular structure left inside the lumen of a duct to relieve an obstruction. Referring to FIGS. 4-5, there is illustrated a stent 100 that is manufactured in accordance with the present invention. As shown in FIG. 4, the stent 100 comprises a plurality of hoop components 102 interconnected by a plurality of flexible connectors 104. The hoop components 102 are formed as a continuous series of substantially longitudinally or axially oriented radial strut members 106 and alternating substantially circumferentially oriented radial arc members 108. Although shown in planar view, the hoop components 102 are essentially ring members that are linked together by the flexible connectors 104 to form a substantially tubular stent structure. The combination of radial strut members 106 and alternating radial arc members 108 form a generally sinusoidal pattern but other patterns may be formed such as a zig-zag pattern by connecting radial strut members directly together. Although the hoop components 102 may be designed with any number of features and assume any number of configurations, in the exemplary embodiment, the radial strut members 106 are wider in their central regions 110. This design feature may be utilized for a number of purposes, including, increased surface area that assists in delivering a therapeutic agent, as discussed in greater detail below.

As shown in FIG. 4A, the flexible connectors 104 are formed from a continuous series of flexible strut members 112 and alternating flexible arc members 114. The flexible connectors 104, as described above, connect adjacent hoop components 102 together. In this exemplary embodiment, the flexible connectors 104 have a substantially N-shape with one end being connected to a radial arc member on one hoop component and the other end being connected to a radial arc member on an adjacent hoop component. As with the hoop components 102, the flexible connectors 104 may comprise any number of design features and any number of configurations. In the exemplary embodiment, the ends of the flexible connectors 104 are connected to different portions of the radial arc members of adjacent hoop components for ease of nesting during crimping of the stent. It is interesting to note that with this exemplary configuration, the radial arcs on adjacent hoop components are slightly out of phase, while the radial arcs on every other hoop component are substantially in phase. In addition, it is important to note that not every radial arc on each hoop component need be connected to every radial arc on the adjacent hoop component.

A wide variety of designs may be utilized for the flexible connectors or connectors in an intraluminal scaffold or stent. For example, in the design described above, the connector comprises two elements, substantially longitudinally oriented strut members and flexible arc members. In alternate designs, however, the connectors may comprise only a substantially longitudinally oriented strut member and no flexible arc member or a flexible arc connector and no substantially longitudinally oriented strut member.

The substantially tubular structure of the stent 100 provides either temporary or permanent scaffolding for maintaining patency of body conduits, such as arteries. The stent 100 comprises a luminal surface and an abluminal surface. The stent 100 is usually inserted into the lumen of a body conduit in a non-expanded form and are then expanded autonomously (or with the aid of a second device) in situ. When used in coronary artery procedures for relieving stenosis, stents are placed percutaneously through the femoral artery. In this type of procedure, the stent 100 is delivered on a catheter and are either self-expanding or, in the majority of cases, expanded by a balloon.

It should be understood that the present invention may be utilized not only in connection with an expandable intraluminal vascular graft for expanding partially occluded segments of a blood vessel, duct or body passageways, such as within an organ, but may so be utilized for many other purposes as an expandable prosthesis for many other types of body passageways. For example, expandable prostheses may also be used for such purposes as: (1) supportive graft placement within blocked arteries opened by transluminal recanalization, but which are likely to collapse in the absence of internal support; (2) similar use following catheter passage through mediastinal and other veins occluded by inoperable cancers; (3) reinforcement of catheter created intrahepatic communications between portal and hepatic veins in patients suffering from portal hypertension; (4) supportive graft placement of narrowing of the esophagus, the intestine, the ureters, the uretha, etc.; (5) intraluminally bypassing a defect such as an aneurysm or blockage within a vessel or organ; and (6) supportive graft reinforcement of reopened and previously obstructed bile ducts. Accordingly, use of the term “prosthesis” encompasses the foregoing usages within various types of body passageways, and the use of the term “intraluminal graft” encompasses use for expanding the lumen of a body passageway. Further in this regard, the term “body passageway” encompasses any lumen or duct within the human body, such as those previously described, as well as any vein, artery, or blood vessel within the human vascular system.

The stent 100 is employed by first identifying a location, for example, a site within the vessel in a patient's body, for deployment of the stent 100. Upon identifying the desired deployment location, for example a stenotic lesion or vulnerable plaque site, a delivery device, such as a catheter carrying the stent 100 crimped to a distal end of the catheter such that the stent 100 is in its closed configuration, is inserted within the vessel in the patient's body. The catheter is used to traverse the vessel until reaching the desired location (site) wherein the distal end of the catheter is positioned at the desired location (site), for instance the lesion, within the vessel. At this point, the stent 100 is deployed to its open configuration by expanding the stent 100 such as by inflation if the stent 100 is a balloon expandable stent or by uncovering or release of the stent 100 if the stent 100 is a self-expanding (crush recoverable) type stent. If a cover is utilized to further protect and secure the stent 100 to the catheter distal end when the stent 100 is a self-expanding stent, the cover is removed from the distal end of the catheter prior to expansion of the stent 100, for instance, through use of an expandable member such as an inflatable balloon. Upon expanding the stent 100 to its open configuration, the expandable member (balloon) is then collapsed, for instance through deflation of the expandable member, whereby the catheter is removed from the deployment site of the vessel and patient's body altogether.

The stent 100 can be delivered by balloon expansion; self-expansion; or a balloon assist self expansion delivery system. The benefit of using the combination system is that stent 100 is not crimped to lower profiles and upon deployment the stent will self expand to a certain value and can be further expanded to the desired dimension by balloon expansion.

Once the stent is in place, the conduit should assume a modeled shape that ensures the proper flow of fluids there through. Nonetheless, additional procedures may be required at other locations downstream from the location where the stent has been placed. In performing these procedures, the presence of pre-placed stents must be taken into consideration as the stent must be passed through to reach the downstream site. Thus, it is advantageous to manufacture a stent from a biodegradable substance, such as a polymer. Polymeric stents, however, may not prevent restenosis as a result of elastic recoil of the polymeric materials. As mentioned previously, the unique design of the stent 100 allows for a wide array of materials, not previously used with prior art stents, to be used with the stent 100 in accordance with the present invention. These include materials normally prone to crushing, deformation or recoil upon deployment of the stent. These materials include plastics and polymers to include biodegradable polymers such as drug eluting polymers.

In general, the stent 100 can be constructed from biodegradable or bioabsorbable polymer compositions. The type of polymers used can degrade via different mechanisms such as bulk or surface erosion. Bulk erodible polymers include aliphatic polyesters such poly (lactic acid); poly (glycolic acid); poly (caprolactone); poly (p-dioxanone) and poly (trimethylene carbonate); and their copolymers and blends. Other polymers can include amino acid derived polymers; phosphorous containing polymers [e.g., poly (phosphoesters)] and poly (ester amide). Surface erodible polymers include polyanhydrides and polyorthoesters. The stent 100 can be made from combinations of bulk and surface erodible polymers to control the degradation mechanism of the stent. For example, the regions that are under high stress can be made from a polymer that will retain strength for longer periods of time, as these will degrade earlier than other regions with low stress. The selection of the polymers will determine the absorption of stents 100 that can be very short (few weeks) and long (weeks to months).

The stents 100 are generally fabricated from forms, such as tubes, made from the polymers, blends, additives and agents described above. The tubes are processed, for example, by laser cutting to form the stent 100. Tubes used to prepare bioabsorbable stents 100 can be fabricated either by melt or solvent processing. The preferred method will be solvent processing, especially for the stents that will contain drug. These tubes can be converted to the desired design by excimer laser processing. Other methods to fabricate the tubes from which the stent is crafted can be injection molding using supercritical fluids such as carbon dioxide.

Different processes may provide different structures, geometries or configurations to the bioabsorbable polymer being processed. For example, tubes processed from rigid polymers tend to be very stiff, but may be very flexible when processed via electrostatic processing or lyophilization. In the former case, the tubes are solid, whereas in the latter case, the tubes are porous. Other processes provide additional geometries and structures that may include fibers, microfibers, thin and thick films, discs, foams, microspheres and even more intricate geometries or configurations. Melt or solution spun fibers, films and tubes may be further processed into different designs such as tubular, slide and lock, helical or otherwise by braiding and/or laser cutting. The differences in structures, geometries or configurations provided by the different processes are useful for preparing different drug delivery devices with desired dimensions, strengths, drug delivery and visualization characteristics. The fibers, films or tubes may be laser cut to a desired geometry or configuration such as in the shape of a stent. Other machining techniques may also be utilized.

As described in greater detail below, the bioabsorbable compositions to prepare the stent 100 will also include agents that enhance the performance of the stent 100 such as therapeutic drugs, radiopaque materials, and other materials, devices or the like. The amount of drug can range from about 1 to 30 percent as an example, although the amount of drug loading can comprise any desired percentage. The stent 100 will carry more drug than a polymer-coated stent. The drug will release by diffusion and during degradation of the stent 100. The amount of drug release will be for a longer period of time to treat local and diffuse lesions; and for regional delivery for arterial branches to treat diseases such as vulnerable plaque. Radiopaque additives can include barium sulfate and bismuth subcarbonate and the amount can be from 5 to 30 percent as an example. Other radiopaque materials include gold particles and iodine compounds. The particle size of these radiopaque materials can vary from nanometers to microns. The benefit of small particle size is to avoid any reduction in the mechanical properties and to improve the toughness values of the devices. Upon polymer absorption, small particles will also not have any adverse effects on surrounding tissues.

Although it is desirable to have therapeutic and radiopaque agents within the polymer, the presence of these agents can adversely affect the mechanical properties of the polymeric device 100. Typically, polymer medical devices are constructed by cutting a desired device from a tube of polymeric material. As discussed above, the polymeric materials used to create the tube can be formed with various agents and additives mixed with the polymer. In accordance with the present invention, the agents or additives are localized in discrete regions of a polymer structure, such as a tube, from which the medical device 100 will be formed in order to minimize the impact on mechanical performance and enhance other performance features of the device 100 such as visibility.

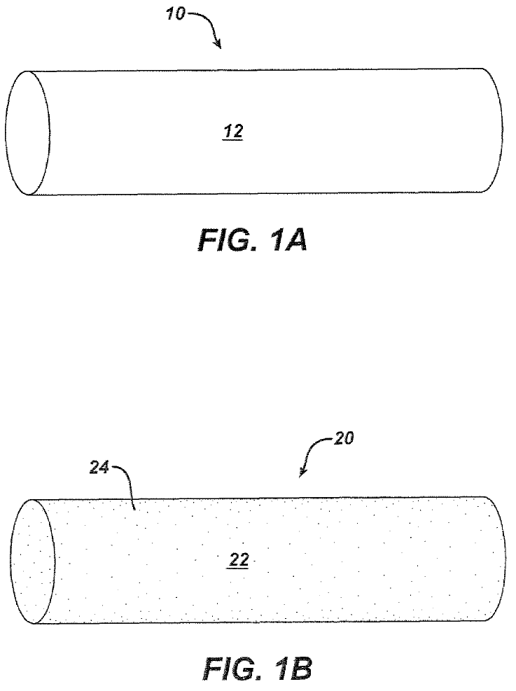

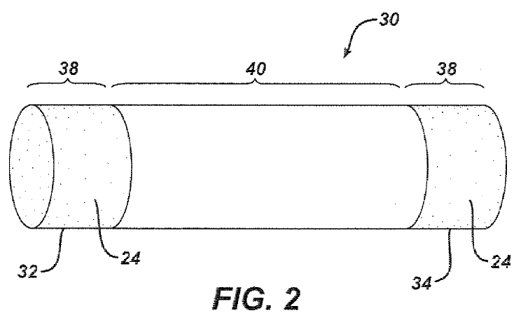

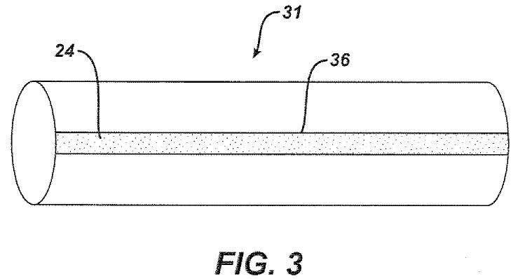

FIG. 1A shows a tube 10 constructed from a polymeric material or a blend of polymeric materials. Tube 10 has a body 12 that can be formed by extrusion or solution processing. A tube can be formed from a polymeric material with an additive and/or agent therein as described in detail below. For example, as seen in FIG. 1B a tube is constructed from a polymer with a radiopaque agent 24 within the body 22 of tube 20. Alternatively, tube 20 may have a therapeutic agent, not shown in the Figures. As shown in FIG. 2, tubes 10 and 20 can be combined such that a unitary tube 30 is produced wherein the polymer containing agent 24 is confined to the distal 32 and proximal 34 regions of the tube 30. The present invention contemplates a wide arrangement of regions of tube 30 where a polymer having agent 24 can be confined or located. For example, agent 24 may be confined to a single point or to a region forming a zig-zag pattern along the length of tube 30. As shown in FIG. 3, a tube 31 may be formed such that agent 24 is confined to a region 36 along the longitudinal length of tube 31. The tubes 30 and 31 have discrete transition regions demarcating the agent 24 from the regions not containing the agent but do not exhibit dimensional discontinuity. If the tubes 30 and 31 also contain a therapeutic agent, the loading of the therapeutic agent may be varied.

In accordance with the present invention tubes having discrete regions are generally formed from polymer structures such as tubes 10 and 20 wherein one tube 10 is formed without an agent and one tube 10 is formed with an agent 24. The tubes 10 and 20 are formed, sized, and then assembled by bonding appropriate segments. The bonding process employs a bonding or gluing agent. Other bonding or sealing processes may be employed such as localized heating and solvent vapor bonding. The localized heating process is performed within a short time period and in a small local area to minimize detrimental effects on therapeutic agents if present. Alternatively, an agent 24 can be placed on a mandrel or other substrate, not shown in the Figures. A polymer tube 10 is advanced onto the substrate in such a way as to bring a discrete region of the tube 10 into contact with the agent 24. The agent 24 is compressed between a stop and the form. After a length of time, the agent 24 bonds with the polymer of tube 10. Other approaches include laying down a coating on a mandrel or substrate using a nozzle, advancing a polymer form such as a tube along the longitudinal length of the mandrel, toward the nozzle, and removing the nozzle when it comes into contact with the polymer form.

It is important to note that it may be desirable to have an agent present throughout the entire tube from which the medical device will be formed, but in different quantities. Alternatively, it may be desirable to have a plurality of discrete regions each having a different agent contained therein. This may be accomplished by forming any number of tubes, depending on the discrete regions to be formed, each having one or more agents contained therein or differing amounts of agent contained therein. Each of the tubes are sized and bonded together to form the tube from which the medical device will be constructed. For purposes of illustration only, the present invention is described with reference to two tubes 10 and 20.

An example of a process employed to construct tube 30 and device 100 there from includes preparing tubes 10 and 20 using either melt extrusion or solvent casting process. Tube 20, containing agent 24, was prepared from PLGA with 10% citrate ester and 30% barium sulfate and tube 10 was prepared from PLGA with 10% citrate ester without barium sulfate from a solvent cast process. Tubes 10 or 20 could also be fabricated to contain therapeutic agents. The tubes 10 and 20 were dried at low drying temperatures (for example, ambient temperature up to 60° C.) to obtain a solvent level of about 20%. Solvent remains in the tubes 10 and 20 as completely dried tubes may not form adequate adhesion during the bonding process. Tubes 10 and 20 are cut to desired size (for example, 16 mm tube segment 40 cut from tube 10 without agent 24; and at least one 2 mm tube segment 38 cut from the tube 20 with agent 24). The segments 38 and 40 are placed on a mandrel in the following order: segment 38 with agent 24 followed by segment 40 without agent 24 (16 mm) optionally followed by another segment 38. Segments 38 (if multiple segments 38 are employed) need not be the same size or even shape and are shown as such for ease of illustrating an aspect of the present invention. A small gap of about 1 mm is left between the segments 38 and 40. These gaps are filled with a bonding medium using a dilute PLGA solution with about 4 to 8% polymer concentration. The bonding medium may also include agents such as therapeutic agents or radiopaque materials and be constructed from the same material as either tube segments 38 or 40 are constructed from.

The segments 38 and 40 are brought into contact and excess solution squeezed from the gaps is removed. The segments 38 and 40 are allowed to dry in a nitrogen atmosphere at ambient temperature, and were then dried at 60° C. or at a suitable temperature to remove remaining solvent to prepare a good bond between the tube and the marker bands. Tube 30 can be prepared in this manner with the proximal and distal ends containing agent 24, such as 30% barium sulfate. Alternatively, a tube such as 31 can be prepared by removing a longitudinal segment from tube 10 and replacing it with a segment from tube 20 that has been sized to fit where the segment was removed from tube 10. Other patterns and designs for the localized regions can be formed in a similar manner as will be appreciated by one of skill in the art. As shown in FIGS. 4 and 4A a medical device such as a stent 100 can be cut or etched from tube 30, for example, by a laser cutting process or acid etching process. Agent 24 is localized to the ends of stent 100. Similarly, a stent 101, a segment of which is shown in FIG. 5, was prepared from tube 31 and has agent 24 localized to a region along the longitudinal length of the stent 101.

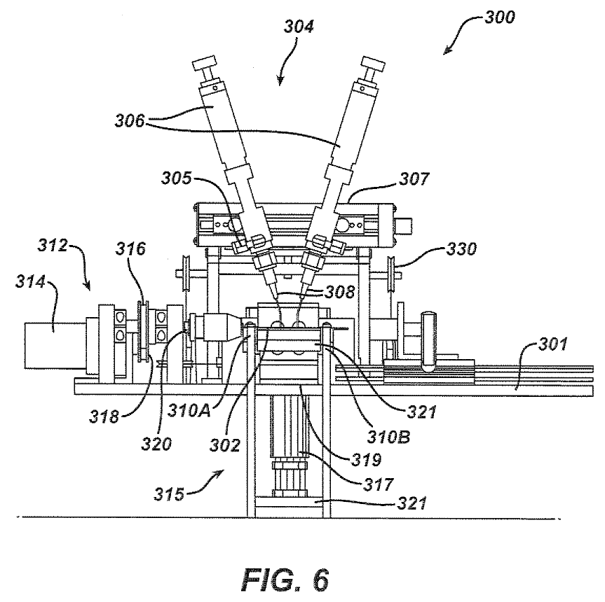

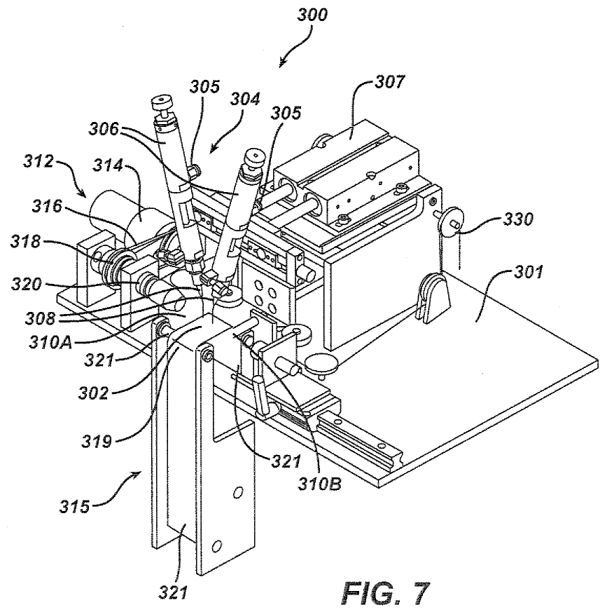

FIGS. 6 and 7 show an apparatus 300 that prepares the tubes with agent 24 localized to a desired region. The apparatus 300 is mounted on a base 301 and includes a mandrel or substrate 302 for mounting the tube segments 38, 40 thereon, a bonding agent dispenser 304, a gripping device 310 for holding polymer forms such as tube segments 38 and 40 onto the mandrel or substrate 302 and pressing them together, and a controller, not shown in the drawings which allows for manual and automatic actuation of the process for joining the segments together. Mandrel 302 is rotated by a drive mechanism 312 mounted onto base 301. The drive mechanism 312 may comprise a variable speed motor 314 connected to a belt 316 and pulley 318 that is mounted onto drive shaft 320. The drive mechanism 312 can be mounted such that it can be translated longitudinally. For example, the drive mechanism may be pushed in a direction towards mandrel 302 thereby translating mandrel 302 longitudinally. Alternatively, mandrel 302 can be mounted to slide along drive shaft 320. This allows for bonding agents to be applied to a location on the mandrel where the at least two segments 38 and 40 will be joined. Preferably, bonding solution-dispensing system 304, described below, can be translated along mandrel 302 when applying bonding agents.

Bonding solution-dispensing system 304 deposits the bonding solution onto the mandrel using at least one, but preferably two dispensers 306. The tips or heads 308 of the dispensers 306 are extended into position using guided pneumatic cylinder 307. The tips or heads 308 can be adjusted forward/backward, up/down and together/apart using adjusting screws to achieve proper location over the mandrel 302. In one embodiment of the present invention, dispenser 306 comprises a pneumatic syringe. Alternatively, the bonding agent may be dispensed manually from a container. A fitting 305 allows for a conduit to be connected that links dispenser 306 to a supply source for bonding solution.

Clamping fingers 310A and 310B pull together the segments 38 and 40 onto the mandrel 302 after the bonding solution has been deposited. The operator can actuate a crank 330 connected to the fingers through a pulley system. Alternatively, a pneumatic drive connected to fingers 310A and 310B can be employed to pull the segments together in the desired direction. For example, the load can be applied to the segments 38 and 40 when a pneumatic cylinder is retracted allowing the fingers to be pulled together on a linear guide. Fittings, not shown in the drawings, are placed onto the mandrel 302 prior to mounting on the apparatus 300 to engage the fingers 310A and 310B. This aids in clamping the tube segments 38 and 40 together after deposition of the bonding solution.

After the segments 38 and 40 are pressed together excess bonding agent will be present on the joined together segments. A wiping assembly 315 removes the excess bonding agent. The assembly 315 comprises a wiping film 319, such as low-density polyethylene film, that is mounted on rollers 321. For example, the film 319 wraps from one roller through the idle rollers and is wound onto another driven roller. As the film 321 is pulled over the rollers, a pneumatic cylinder 317 lifts the front roller so that the film 319 is pushed into contact with the joined tube segments mounted on mandrel 302. The film 319 wipes off the excess bonding solution.

Although described in use for manufacturing stents, the apparatus 300 and method employed thereby can be used for different types of devices besides stents such as shunts, valves, filters and the like. Different types of therapeutic agents can be used in a single device by varying the amount and composition of the regions to which the agents are localized. Finally, tubes can be formed with agent dispersed in the polymer matrix, chemically attached to the polymer backbone, or coated onto the polymer.

Other Properties of Polymeric Devices

Polymeric materials may be broadly classified as synthetic, natural and/or blends thereof. Within these broad classes, the materials may be defined as biostable or biodegradable. Examples of biostable polymers include polyolefins, polyamides, polyesters, fluoropolymers, and acrylics. Examples of natural polymers include polysaccharides and proteins.

The drug delivery devices according to the systems and methods of the present invention may be disease specific, and may be designed for local or regional therapy, or a combination thereof. They may be used to treat coronary and peripheral diseases such as vulnerable plaque, restenosis, bifurcated lesions, superficial femoral artery, below the knee, saphenous vein graft, arterial tree, small and tortuous vessels, and diffused lesions. The drugs or other agents delivered by the drug delivery devices according to the systems and methods of the present invention may be one or more drugs, bio-active agents such as growth factors or other agents, or combinations thereof. The drugs or other agents of the device are ideally controllably released from the device, wherein the rate of release depends on either or both of the degradation rates of the bioabsorbable polymers comprising the device and the nature of the drugs or other agents. The rate of release can thus vary from minutes to years as desired.

Bioabsorbable and/or biodegradable polymers consist of bulk and surface erodable materials. Surface erosion polymers are typically hydrophobic with water labile linkages. Hydrolysis tends to occur fast on the surface of such surface erosion polymers with no water penetration in bulk. The initial strength of such surface erosion polymers tends to be low however, and often such surface erosion polymers are not readily available commercially. Nevertheless, examples of surface erosion polymers include polyanhydrides such as poly (carboxyphenoxy hexane-sebacic acid), poly (fumaric acid-sebacic acid), poly (carboxyphenoxy hexane-sebacic acid), poly (imide-sebacic acid)(50-50), poly (imide-carboxyphenoxy hexane) (33-67), and polyorthoesters (diketene acetal based polymers).

Bulk erosion polymers, on the other hand, are typically hydrophilic with water labile linkages. Hydrolysis of bulk erosion polymers tends to occur at more uniform rates across the polymer matrix of the device. Bulk erosion polymers exhibit superior initial strength and are readily available commercially.

Examples of bulk erosion polymers include poly (α-hydroxy esters) such as poly (lactic acid), poly (glycolic acid), poly (caprolactone), poly (p-dioxanone), poly (trimethylene carbonate), poly (oxaesters), poly (oxaamides), and their co-polymers and blends. Some commercially readily available bulk erosion polymers and their commonly associated medical applications include poly (dioxanone) [PDS® suture available from Ethicon, Inc., Somerville, N.J.], poly (glycolide) [Dexon® sutures available from United States Surgical Corporation, North Haven, Conn.], poly (lactide)-PLLA [bone repair], poly (lactide/glycolide) [Vicryl® (10/90) and Panacryl® (95/5) sutures available from Ethicon, Inc., Somerville, N.J.], poly (glycolide/caprolactone (75/25) [Monocryl® sutures available from Ethicon, Inc., Somerville, N.J.], and poly (glycolide/trimethylene carbonate) [Maxon® sutures available from United States Surgical Corporation, North Haven, Conn.].

Other bulk erosion polymers are tyrosine derived poly amino acid [examples: poly (DTH carbonates), poly (arylates), and poly (imino-carbonates)], phosphorous containing polymers [examples: poly (phosphoesters) and poly (phosphazenes)], poly (ethylene glycol) [PEG] based block co-polymers [PEG-PLA, PEG-poly (propylene glycol), PEG-poly (butylene terephthalate)], poly (α-malic acid), poly (ester amide), and polyalkanoates [examples: poly (hydroxybutyrate (HB) and poly (hydroxyvalerate) (HV) co-polymers].

Of course, the devices may be made from combinations of surface and bulk erosion polymers in order to achieve desired physical properties and to control the degradation mechanism. For example, two or more polymers may be blended in order to achieve desired physical properties and device degradation rate. Alternately, the device may be made from a bulk erosion polymer that is coated with a surface erosion polymer. The drug delivery device may be made from a bulk erosion polymer that is coated with a drug containing a surface erosion polymer. For example, the drug coating may be sufficiently thick that high drug loads may be achieved, and the bulk erosion polymer may be made sufficiently thick that the mechanical properties of the device are maintained even after all of the drug has been delivered and the surface eroded.

Shape memory polymers may also be used. Shape memory polymers are characterized as phase segregated linear block co-polymers having a hard segment and a soft segment. The hard segment is typically crystalline with a defined melting point, and the soft segment is typically amorphous with a defined glass transition temperature. The transition temperature of the soft segment is substantially less than the transition temperature of the hard segment in shape memory polymers. A shape in the shape memory polymer is memorized in the hard and soft segments of the shape memory polymer by heating and cooling techniques. Shape memory polymers may be biostable and bioabsorbable. Bioabsorbable shape memory polymers are relatively new and comprise thermoplastic and thermoset materials. Shape memory thermoset materials may include poly (caprolactone)dimethylacrylates, and shape memory thermoplastic materials may include poly (caprolactone) as the soft segment and poly (glycolide) as the hard segment.

In order to provide materials with high toughness, such as is often required for orthopedic implants, sutures, stents, grafts and other medical applications including drug delivery devices, the bioabsorbable polymeric materials may be modified to form composites or blends thereof. Such composites or blends may be achieved by changing either the chemical structure of the polymer backbone, or by creating composite structures by blending them with different polymers and plasticizers. The addition of plasticizers, which are generally low molecular weight materials, or a soft (lower glass transition temperature) miscible polymer, will depress the glass transition temperature of the matrix polymer system. In general, these additional materials that are used to modify the underlying bioabsorbable polymer should preferably be miscible with the main matrix polymer system to be effective.

Plasticizers that are added to the matrix of bioabsorbable polymer materials will make the device more flexible and typically reduces the processing temperatures in case of processing materials in melt. The plasticizers are added to the bioabsorbable materials of the device prior to or during processing thereof. As a result, degradation of drugs incorporated into the bioabsorbable materials having plasticizers added thereto during processing is further minimized.

Plasticizers or mixtures thereof suitable for use in the present invention may be selected from a variety of materials including organic plasticizers and those like water that do not contain organic compounds. Organic plasticizers include but not limited to, phthalate derivatives such as dimethyl, diethyl and dibutyl phthalate; polyethylene glycols with molecular weights preferably from about 200 to 6,000, glycerol, glycols such as polypropylene, propylene, polyethylene and ethylene glycol; citrate esters such as tributyl, triethyl, triacetyl, acetyl triethyl, and acetyl tributyl citrates, surfactants such as sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof. Preferred organic plasticizers include citrate esters; polyethylene glycols and dioxane.

Polymer blends are commonly prepared to achieve the desired final polymer properties. In accordance with the present invention, polymer blends are prepared to increase the elongation at break values or ultimate strain and thereby improving the toughness of the material that will be used to prepare vascular devices such as stents. Selection of the materials is important in order to achieve high toughness values of the matrix polymer. Matching solubility parameters and increase in free volume is important for the polymer blends to achieve the desired performance. The main difference between adding a plasticizer and a polymer to the matrix polymer is the difference in their molecular weights. As mentioned earlier, plasticizers have lower molecular weight compared to a polymeric additive. However, some low molecular weight polymers may also be used as a plasticizer. It is possible to achieve high toughness values by adding low amounts of plasticizer compared to a polymeric additive. Relatively high molecular weight material has been used as the matrix material for the present invention. For example, the molecular weight (weight average) of PLGA resins may be above 300,000 Daltons. Thermodynamically, molecular weight plays a big role in miscibility of polymer systems. There is higher miscibility between polymer and a low molecular weight additive compared to a high molecular weight additive. As mentioned earlier, the addition of a miscible polymer will lower glass transition temperature, decrease modulus and tensile strength with an increase in the toughness values.

As an example of producing a composite or blended material, blending a stiff polymer such as poly (lactic acid), poly (glycolide) and poly (lactide-co-glycolide) copolymers with a soft and elastomeric polymer such as poly (caprolactone) and poly (dioxanone) tends to produce a material with high toughness and high stiffness. An elastomeric co-polymer may also be synthesized from a stiff polymer and a soft polymer in different ratios. For example, poly (glycolide) or poly (lactide) may be copolymerized with poly (caprolactone) or poly (dioxanone) to prepare poly (glycolide-co-caprolactone) or poly (glycolide-co-dioxanone) and poly (lactide-co-caprolactone) or poly (lactide-co-dioxanone) copolymers. These elastomeric copolymers may then be blended with stiff materials such as poly (lactide), poly (glycolide) and poly (lactide-co-glycolide) copolymers to produce a material with high toughness and ductility. Alternatively, terpolymers may also be prepared from different monomers to achieve desired properties. For example, poly (caprolactone-co-glycolide-co-lactide) may be prepared in different ratios.

In addition to increasing the toughness values with the addition of the soft polymers, the absorption time may also be modified. For example, the blend of PLGA with polycaprolactone will increase the total absorption time of the blended material as polycaprolactone degrades slower than PLGA. The total absorption may be reduced for PLGA by blending it with faster degrading materials such as poly (dioxanone) and their copolymers with poly (glycolide) and poly (lactide); and copolymers of poly (glycolide) such as poly (caprolactone-co-glycolide). Reinforced composites may also be prepared by blending high modulus PGA fibers or bioabsorbable particulate fillers with PLGA to form composites in the presence of the plasticizers or soft materials to improve the modulus of the final material.

Melt blends of polymers, with melting points lower than the melting point of the bioabsorbable materials in which the drugs or other bio-active agents are to be incorporated, may also be added to the bioabsorbable materials that are to comprise the device. Adding the blends of polymers having the lower melting points also helps to reduce processing temperatures and minimize degradation of the drugs or agents thereby.

It is important to note that the drug or therapeutic agent, in sufficient concentration, may be used as an additive for modifying the polymer properties. In other words, the drug or therapeutic agent may be utilized as part of the blend, rather than as a material affixed to a base material, similar to the blends described herein to achieve the desired end product properties in addition to providing a therapeutic effect.

Radiopaque materials may be added to the polymer blend from which the device is constructed to ensure visualization of the device as it is implanted in the patient. The radiopaque materials may be added directly to the matrix of bioabsorbable materials comprising the device during processing thereof resulting in fairly uniform incorporation of the radiopaque materials throughout the device. Alternately, the radiopaque materials may be added to the device in the form of a layer, a coating, a mark or band or powder at designated portions of the device depending on the geometry of the device and the process used to form the device. Coatings may be applied to the device in a variety of processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques. Such coatings sometimes have less negative impact on the physical characteristics (eg., size, weight, stiffness, flexibility) and performance of the device than do other techniques.

Preferably, the radiopaque material does not add significant stiffness to the device so that the device may readily traverse the anatomy within which it is deployed. The radiopaque material should be biocompatible with the tissue within which the device is deployed. Such biocompatibility minimizes the likelihood of undesirable tissue reactions with the device. Inert noble metals such as gold, platinum, iridium, palladium, and rhodium are well-recognized biocompatible radiopaque materials. Other radiopaque materials include barium sulfate (BaSO₄), bismuth subcarbonate [(BiO)₂CO3] and bismuth oxide. Preferably, the radiopaque materials adhere well to the device such that peeling or delamination of the radiopaque material from the device is minimized, or ideally does not occur. Where the radiopaque materials are added to the device as metal bands, the metal bands may be crimped at designated sections of the device. Alternately, designated sections of the device may be coated with a radiopaque metal powder, whereas other portions of the device are free from the metal powder.

Delivery of Therapeutic Agents

The components of the stent 100 described, i.e. hoops, loops, struts and flexible links, may have drug coatings, drug and polymer coating combinations, and/or drug dispersed throughout the polymer that is used to fabricate the stent that are used to deliver drugs, i.e. therapeutic and/or pharmaceutical agents including: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP)II_{b}IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); antiinflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF) platelet derived growth factor (PDGF), erythropoetin; angiotensin receptor blocker; nitric oxide donors; anti-sense oligionucleotides and combinations thereof; cell cycle inhibitors, mTOR inhibitors, and growth factor signal transduction kinase inhibitors. It is important to note that one or more of the lattice components (e.g. hoops, loops, struts and flexible links) are coated with one or more of the drug coatings or drug and polymer coating combinations. Additionally, as mentioned above, the stent 100 is alternatively made of a polymer material itself such as a biodegradable material capable of containing and eluting one or more drugs, in any combination, in accordance with a specific or desired drug release profile.

The local delivery of therapeutic agent/therapeutic agent combinations may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach.

Devices that serve to improve the structure and function of tissue or organ may also show benefits when combined with the appropriate agent or agents. For example, improved osteointegration of orthopedic devices to enhance stabilization of the implanted device could potentially be achieved by combining it with agents such as bone-morphogenic protein. Similarly other surgical devices, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, including stents, stent-grafts and other devices for repairing aneurysims, and vascular supports could also provide enhanced patient benefit using this drug-device combination approach. Perivascular wraps may be particularly advantageous, alone or in combination with other medical devices. The perivascular wraps may supply additional drugs to a treatment site. Essentially, any other type of medical device may be coated in some fashion with a drug or drug combination, which enhances treatment over use of the singular use of the device or pharmaceutical agent.

Different drugs may be utilized as therapeutic agents, including sirolimus, heparin, everolimus, tacrolimus, paclitaxel, cladribine as well as classes of drugs such as statins. These drugs and/or agents may be hydrophilic, hydrophobic, lipophilic and/or lipophobic. The type of agent will play a role in determining the type of polymer. The amount of the drug in the coating may be varied depending on a number of factors including, the storage capacity of the coating, the drug, the concentration of the drug, the elution rate of the drug as well as a number of additional factors. The amount of drug may vary from substantially zero percent to substantially one hundred percent. Typical ranges may be from about less than one percent to about forty percent or higher. Drug distribution in the coating may be varied. The one or more drugs may be distributed in a single layer, multiple layers, single layer with a diffusion barrier or any combination thereof.

In addition to various medical devices, the coatings on these devices may be used to deliver therapeutic and pharmaceutic agents including, all the compounds described above and anti-proliferative agents, anti-throrombogenic agents, anti-restenotic agents, anti-infective agents, anti-viral agents, anti-bacterial agents, anti-fungal agnts, anti-inflammatory agents, cytostatic agents, cytotoxic agents, immunosuppressive agents, anti-microbial agents, anti-calcification agents, anti-encrustation agents, statins, hormones, anti-cancer agents, anti-coagulants, anti-migrating agents and tissue growth promoting agents.

As described herein, various drugs or agents may be incorporated into the medical device by a number of mechanisms, including blending it with the polymeric materials or affixing it to the surface of the device. Different drugs may be utilized as therapeutic agents, as described above. These drugs and/or agents may be hydrophilic, hydrophobic, lipophilic and/or lipophobic.

The local delivery of drug/drug combinations from a stent has the following advantages; namely, the prevention of vessel recoil and remodeling through the scaffolding action of the stent and the prevention of multiple components of neointimal hyperplasia or restenosis as well as a reduction in inflammation and thrombosis. This local administration of drugs, agents or compounds to stented coronary arteries may also have additional therapeutic benefit. For example, higher tissue concentrations of the drugs, agents or compounds may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, anti-thrombotic drugs, agents or compounds.

A variety of drugs, agents or compounds may be utilized in combination with any number of medical devices, and in particular, with implantable medical devices such as stents and stent-grafts. Other devices such as vena cava filters and anastomosis devices may be used with coatings having drugs, agents or compounds therein or the devices themselves may be fabricated with polymeric materials that have the drugs contained therein. Any of the stents or other medical devices described herein may be utilized for local or regional drug delivery. Balloon expandable stents may be utilized in any number of vessels or conduits, and are particularly well suited for use in coronary arteries. Self-expanding stents, on the other hand, are particularly well suited for use in vessels where crush recovery is a critical factor, for example, in the carotid artery.

Any of the above-described medical devices may be utilized for the local delivery of drugs, agents and/or compounds to other areas, not immediately around the device itself. In order to avoid the potential complications associated with systemic drug delivery, the medical devices of the present invention may be utilized to deliver therapeutic agents to areas adjacent to the medical device. For example, a rapamycin coated stent may deliver the rapamycin to the tissues surrounding the stent as well as areas upstream of the stent and downstream of the stent (regional delivery). The degree of tissue penetration depends on a number of factors, including the drug, agent or compound, the concentrations of the drug and the release rate of the agent. The same holds true for coated anastomosis devices.

The amount of drugs or other agents incorporated within the drug delivery device according to the systems and methods of the present invention may range from about 0 to 99 percent (percent weight of the device). The drugs or other agents may be incorporated into the device in different ways. For example, the drugs or other agents may be coated onto the device after the device has been formed, wherein the coating is comprised of bioabsorbable polymers into which the drugs or other agents are incorporated. Alternately, the drugs or other agents may be incorporated into the matrix of bioabsorbable materials comprising the device. The drugs or agents incorporated into the matrix of bioabsorbable polymers may be in an amount the same as, or different than, the amount of drugs or agents provided in the coating techniques discussed earlier if desired. These various techniques of incorporating drugs or other agents into, or onto, the drug delivery device may also be combined to optimize performance of the device, and to help control the release of the drugs or other agents from the device.

Where the drug or agent is incorporated into the matrix of bioabsorbable polymers comprising the device, for example, the drug or agent will release by diffusion and during degradation of the device. The amount of drug or agent released by diffusion will tend to release for a longer period of time than occurs using coating techniques, and may often more effectively treat local and diffuse lesions or conditions thereof. For regional drug or agent delivery such diffusion release of the drugs or agents is effective as well. Polymer compositions and their diffusion and absorption characteristics will control drug elution profile for these devices. The drug release kinetics will be controlled by drug diffusion and polymer absorption. Initially, most of the drug will be released by diffusion from the device surfaces and bulk and will then gradually transition to drug release due to polymer absorption. There may be other factors that will also control drug release. If the polymer composition is from the same monomer units (e.g., lactide; glycolide), then the diffusion and absorption characteristics will be more uniform compared to polymers prepared from mixed monomers. Also, if there are layers of different polymers with different drug in each layer, then there will be more controlled release of drug from each layer. There is a possibility of drug present in the device until the polymer fully absorbs thus providing drug release throughout the device life cycle.

The drug delivery device according to the systems and methods of the present invention preferably retains its mechanical integrity during the active drug delivery phase of the device. After drug delivery is achieved, the structure of the device ideally disappears as a result of the bioabsorption of the materials comprising the device. The bioabsorbable materials comprising the drug delivery device are preferably biocompatible with the tissue in which the device is implanted such that tissue interaction with the device is minimized even after the device is deployed within the patient. Minimal inflammation of the tissue in which the device is deployed is likewise preferred even as degradation of the bioabsorbable materials of the device occurs. In order to provide multiple drug therapy, enriched or encapsulated drug particles or capsules may be incorporated in the polymer matrix. Some of these actives may provide different therapeutic benefits such as anti-inflammatory, anti-thrombotic; etc.

In accordance with another exemplary embodiment, the stents described herein, whether constructed from metals or polymers, may be utilized as therapeutic agents or drug delivery devices wherein the drug is affixed to the surface of the device. The devices may be coated with a biostable or bioabsorbable polymer or combinations thereof with the therapeutic agents incorporated therein. Typical material properties for coatings include flexibility, ductility, tackiness, durability, adhesion and cohesion. Biostable and bioabsorbable polymers that exhibit these desired properties include methacrylates, polyurethanes, silicones, poly (vinyl acetate), poly (vinyl alcohol), ethylene vinyl alcohol, poly (vinylidene fluoride), poly (lactic acid), poly (glycolic acid), poly (caprolactone), poly (trimethylene carbonate), poly (dioxanone), polyorthoester, polyanhydrides, polyphosphoester, polyaminoacids as well as their copolymers and blends thereof.

As described above, polymer stents may contain therapeutic agents as a coating, e.g. a surface modification. Alternatively, the therapeutic agents may be incorporated into the stent structure, e.g. a bulk modification that may not require a coating. For stents prepared from biostable and/or bioabsorbable polymers, the coating, if used, could be either biostable or bioabsorbable. However, as stated above, no coating may be necessary because the device itself is fabricated from a delivery depot. This embodiment offers a number of advantages. For example, higher concentrations of the therapeutic agent or agents may be achievable such as about >50 percent by weight. In addition, with higher concentrations of therapeutic agent or agents, regional drug delivery (>5 mm) is achievable for greater durations of time. This can treat different lesions such as diffused lesions, bifurcated lesions, small and tortuous vessels, and vulnerable plaque. Since these drug loaded stents or other devices have very low deployment pressures (3 to 12 atmospheres), it will not injure the diseased vessels. These drug-loaded stents can be delivered by different delivery systems such balloon expandable; self-expandable or balloon assist self-expanding systems.

Although the present invention has been described above with respect to particular preferred embodiments, it will be apparent to those skilled in the art that numerous modifications and variations can be made to these designs without departing from the spirit or essential attributes of the present invention. Accordingly, reference should be made to the appended claims, rather than to the foregoing specification, as indicating the scope of the invention. The descriptions provided are for illustrative purposes and are not intended to limit the invention nor are they intended in any way to restrict the scope, field of use or constitute any manifest words of exclusion.

What is claimed is:

1. An apparatus comprising:
a lattice of interconnected elements constructed from a structure having at least one distinct region having an agent distributed throughout the region;
wherein the structure is constructed from a plurality of bodies formed from at least one polymeric material;
wherein at least one of the plurality of bodies includes an agent.

2. The apparatus of claim 1 wherein the plurality of bodies comprises a plurality of polymer tubes.

3. The apparatus of claim 2 wherein at least one of the plurality of tubes is formed from at least one polymer having an agent therein.

4. The apparatus of claim 1 wherein at least one of the plurality of bodies is formed from a different polymer than the other of the plurality of bodies.

5. The apparatus of claim 1 wherein at least one of the plurality of bodies is formed from at least polymeric material having at least one additive.

6. The apparatus of claim 1 wherein at least one of the plurality of bodies is formed from at least one polymer having at least one plasticizer.

7. The apparatus of claim 1 wherein each of the plurality of polymer bodies has an agent and the agent of each of the plurality of polymer bodies is different from the agent of the other of the plurality of polymer bodies.

8. The apparatus of claim 1 wherein each of the plurality of polymer bodies has an agent and each of the plurality of polymer bodies has a different amount of agent than the plurality of polymer bodies.

9. The apparatus of claim 1 wherein the plurality of bodies are cut into segments and joined together to form the structure such that the segments are aligned in a pre-determined manner to form the at least one distinct region.

10. The apparatus of claim 1 wherein the distinct region contains the agent such that the ratio of the amount of agent located on an abluminal surface of the region to the agent contained within the remaining part of the region is greater than 10 percent.

11. The apparatus of claim 9, wherein the structure is cut to form the lattice.

12. The apparatus of claim 11 wherein the lattice comprises a plurality of hoops formed from a plurality of struts connected by a plurality of loops; and a plurality of bridges connecting adjacent hoops.

13. The apparatus of claim 1 wherein the region comprises at least one end of the lattice.

14. The apparatus of claim 1 wherein the agent is a therapeutic agent.

15. The apparatus of claim 1 wherein the agent is a radiopaque agent.

16. An apparatus for making a medical device having at least one discrete region comprising:
a substrate onto which a plurality of segments are mounted in a pre-determined pattern;
a means for moving the substrate;
a means for dispensing a solution onto a desired region of the substrate; and
a means for bringing the plurality of segments into contact with each other and the solution.

17. The apparatus of claim 16 wherein the substrate comprises an elongated mandrel onto which a segment is mounted.

18. The apparatus of claim 16 wherein the means for moving the substrate rotates the substrate.

19. The apparatus of claim 16 wherein the means for moving the substrate comprises a motor having its shaft in driving connection with the substrate.

20. The apparatus of claim 19 wherein the means for moving the substrate is translated along an axis that is parallel to a longitudinal axis of the substrate.

21. The apparatus of claim 16 wherein the means for dispensing a solution onto the substrate is mounted above the substrate so as to move along a longitudinal axis of the mandrel.

22. The apparatus of claim 21 wherein the means for dispensing solution onto the substrate is mounted above the substrate by a guided pneumatic cylinder system that moves the means for dispensing solution relative to the substrate.

23. The apparatus of claim 22 wherein the means for dispensing a solution onto the substrate comprises at least one pneumatic syringe.

24. The apparatus of claim 23 wherein the pneumatic syringe has an outlet port with a nozzle located thereon.

25. The apparatus of claim 16 wherein the means for bringing the plurality of segments into contact with each other comprises a first and second gripping finger mounted along the substrate.

26. The apparatus of claim 25 wherein the gripping fingers are pneumatically driven so as to bring the segments together.

28. The apparatus of claim 16 wherein the solution comprises an adhesive.

29. The apparatus of claim 16 wherein the solution comprises a solvent.

30. The apparatus of claim 16 further comprising a means for removing excess solution.

31. The apparatus of claim 16 wherein the means for removing excess solution comprises a wiping assembly having a frame mounted onto a pneumatic cylinder, a plurality of rollers mounted onto the frame, a motor for driving one of the plurality of rollers, and a length of material mounted onto the rollers.

32. The apparatus of claim 31 wherein the pneumatic cylinder moves the frame in a direction to bring the length of material into contact with the plurality of segments mounted onto the substrate as the motor drives the rollers and length of material so as to wipe away excess solution.

33. An apparatus for making a medical device having at least one discrete region comprising:
a substrate onto which a plurality of segments are mounted in a pre-determined pattern;
a drive that rotates the substrate;
at least one dispenser for placing a bonding solution onto a desired region of the substrate said dispenser being movably mounted on a guided cylinder in proximity to the substrate; and
a plurality of projections for bringing the plurality of segments into contact with each other and the bonding solution.

34. The apparatus of claim 33 wherein the substrate comprises a mandrel.

35. The apparatus of claim 33 wherein the drive comprises a motor that is linked to the substrate by a pulley system.

36. The apparatus of claim 33 wherein the at least one dispenser comprises a syringe having a nozzle at its distal end.

37. The apparatus of claim 33 further comprising a wiping assembly.

38. The apparatus of claim 37 wherein the wiping assembly comprises a frame mounted onto a pneumatic cylinder, a plurality of rollers mounted onto the frame, a motor for driving one of the plurality of rollers, and a length of material mounted onto the rollers.

39. The apparatus of claim 38 wherein the pneumatic cylinder moves the frame in a direction to bring the length of material into contact with the plurality of segments mounted onto the substrate as the motor drives the rollers and length of material so as to wipe away excess solution.

40. The apparatus of claim 33 wherein the plurality of segments is formed from a polymer.

41. The apparatus of claim 40 wherein at least one of the plurality of segments is formed from a polymer having an agent.

42. The apparatus of claim 41 wherein at least one of the plurality of segments is formed from a different polymer than the other of the plurality of segments.

43. The apparatus of claim 41 wherein the agent is a therapeutic agent.

44. The apparatus of claim 41 wherein the agent is a radiopaque agent.

45. The apparatus of claim 33 wherein each of the segments is formed from polymer having an agent wherein each of the plurality of segments has a different agent than the other of the plurality of segments.

46. An apparatus for making a medical device having at least one discrete region comprising:
a substrate onto which a plurality of segments are mounted in a pre-determined pattern;
a means for moving the substrate;
a means for dispensing a solution onto a desired region of the substrate;
a means for bringing the plurality of segments into contact with each other and the solution; and
a means for removing excess solution.
